(19) United States
(12) Patent Application Publication
(10) Pub. No.: US 2017/0234866 A1
(43) Pub. Date: Aug. 17, 2017

(54) MULTIPLEXED LATERAL FLOW ASSAY

(71) Applicant: Massachusetts Institute of Technology, Cambridge, MA (US)

(72) Inventors: Kimberly Hamad-Schifferli, Somerville, MA (US); Irene Bosch, Brookline, MA (US); Chun-Wan Yen, Lansdale, PA (US); Helena de Puig, Cambridge, MA (US); Justina O. Tam, Somerville, MA (US); Lee Gehrke, Cotuit, MA (US)

(21) Appl. No.: 15/041,788

(22) Filed: Feb. 11, 2016

(51) Int. Cl. G01N 33/558; G01N 33/569; G01N 33/543

(52) U.S. Cl. CPC G01N 33/558; G01N 33/54306; G01N 33/56983

(57) ABSTRACT

The present invention provides a lateral flow multiplex assay strip, and lateral flow assay systems and kits comprising the assay strips of the invention and methods of using the assay strip and systems and kits to detect the levels of two or more target analytes that may be present in a liquid sample, wherein the two more target analytes are detectable at a single test area of the assay.

### GOVERNMENT SUPPORT

This invention was made with Government support under grant number R33 AI100190 awarded by National Institutes of Health. The government has certain rights in the invention.

### BACKGROUND OF THE INVENTION

Lateral flow assays (LFA) are immunoassays that can be used to detect biological agents including various analytes in samples that may contain such agents. The general format of LFA uses the same rationale as ELISA, where immobilized capture antibody or is bound onto a solid phase nitrocellulose membrane for example instead of a plastic well. The advantage of the LFA format is that the membrane enables a one-step assay unlike that found in the multiple-step ELISA. Based on the principal of high affinity, sensitivity and selectivity between specific antibody-antigen pairs, immunology-based assays are readily available due to the huge variety of existing antibodies and the potential to produce many more as well as the availability of reasonably priced reaction reagents. Lateral flow technology is well-suited to point-of-care (POC) disease diagnostics because it is robust and inexpensive, without requiring power, a cold chain for storage and transport, or specialized reagents.

Many LFA devices comprise a porous matrix capable of supporting the test and which is made of a material which is capable of absorbing a liquid sample and which promotes capillary action of liquid sample along the matrix, such as nitrocellulose. The matrix may come in any shape or size, one common size being a strip that is capable of being held in a hand. In one preferred test format, after absorbing the liquid sample, such as a biological sample, onto the sample pad, the liquid moves into the conjugate pad by capillary action, rehydrates the conjugated particles labelled with a detectable moiety such as a colored label, allowing for the mixing of these particles with the absorbed liquid sample. The labelled conjugates interact with the specific analyte contained in the sample, thereby initiating the intermolecular interactions, which are dependent on the affinity and avidity of the reagents. Then the labelled conjugate and its specific analyte migrates towards the test line also referred to herein as “test area” capturing and recognizing the binding analyte, where it becomes immobilized and produces a distinct signal for example, in the form of, for example, a colored line, indicating the test is positive. Excess reagents move past the capture lines to an optional control line comprising a positive control that insures that all reagents are functional and finally the excess reagents are entrapped in the wick pad, which is designed to draw the sample across the membrane by capillary action and thereby maintain a lateral flow along the strip.

Some lateral flow assays may have more than one test line for multiplex testing of multiple analytes, but each additional test line greatly increases the complexity of the immunosensor, and thus increases cost. Multiplexing, the detection of more than one marker in a single strip, offers further advantages for increasing speed and lowering costs by screening for multiple pathogens simultaneously. While traditional lateral flow devices such as pregnancy tests screen for a single marker, recent technical advances permit multiplexing by spatial separation of lines on a single strip, or branched flow into separate test areas.

Potential disadvantages of multiplexing include non-specific binding and crossover, leading to false positive results. The shortcomings of the prior art LFAs and prior art multiplexed LFAs has prompted an urgent need for improved LFAs that allow quantified measurements, that are more sensitive and have less false positives within the same sample and miniaturized framework of the current lateral flow assay strip while maintaining a rapid diagnostic, and an easy handling by untrained personnel in the field.

### SUMMARY OF THE INVENTION

The present invention provides lateral flow multiplex assay strips and lateral flow assay systems comprising the assay strips of the invention and methods of using the system to measure the levels of two or more target analytes that may be present in a liquid sample such body fluid, wherein the two more target analytes are detectable in a single test area of the assay.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 illustrates a multiplexed assay strip of the invention comprising a single test area binding two or more conjugated analytes from the liquid sample.

FIG. 2 illustrates a multiplexed assay strip of the invention wherein the assay strip is contacted with a liquid sample comprising labelled detection antibodies. This format is also known as a “dipstick” or “half strip” format.

### DETAILED DESCRIPTION OF THE INVENTION

The terms “a”, “an” and “the” as used herein are defined to mean “one or more” and include the plural unless the context is inappropriate.

As used herein, the term “porous material” refers to a material capable of providing capillary movement or lateral flow. This would include material such as nitrocellulose, nitrocellulose blends with polyester or cellulose, untreated paper, porous paper, rayon, glass fiber, acrylonitrile copolymer or nylon or other porous materials that allow lateral flow. Porous materials useful in the devices described herein permit transit, either through the porous matrix or over the surface of the material, of particle label used in these devices.

By “capillary flow”, it is meant liquid flow in which all of the dissolved or dispersed components of the liquid are carried at substantially equal rates and with relatively unimpaired flow laterally through the membrane, as opposed to preferential retention of one or more components as would occur, e.g., in materials capable of adsorbing or imbibing one or more components.

As used herein, the term “lateral flow” refers to capillary flow through a material in a horizontal direction, but will be understood to apply to the flow of a liquid from a point of application of the liquid to another lateral position even if, for example, the device is vertical or on an incline. Lateral flow depends upon properties of the liquid/substrate interaction (surface wetting or wicking action) and does not require or involve application of outside forces, e.g., vacuum or pressure applications by the user.

As used herein, the term “reagent” encompasses substances which can be suspended or immobilized on a porous membrane or substrate and which contribute to a means for detecting analyte. For example, a “reagent” can permit visual detection of a labeled substance or substances—for example, colored metal nanoparticles, that have been bound indirectly to an analyte of interest. The label may alternatively be detected using instrumentation known to those skilled in the art such as a spectrophotometer or fluorescence detector. The reagents on the porous membrane or substrate may be immobilized or may be diffusible. Alternatively, a reagent may be diffusible such that when contacted with the sample, the reagents become mobile and move with the sample toward the distal end of the test strip.

As is known in the art, an “antibody” is an immunoglobulin that binds specifically to a particular antigen. The term encompasses immunoglobulins that are naturally produced in that they are generated by an organism reacting to the antigen, and also those that are synthetically produced or engineered. An antibody may be monoclonal or polyclonal. An antibody may be a member of any immunoglobulin class, including any of the human classes: IgG, IgM, IgA, and IgD. A typical immunoglobulin (antibody) structural unit as understood in the art, is known to comprise a tetramer. Each tetramer is composed of two identical pairs of polypeptide chains, each pair having one “light” (approximately 25 kD) and one “heavy” chain (approximately 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains respectively. Each variable region is further subdivided into hypervariable (HV) and framework (FR) regions. The hypervariable regions comprise three areas of hypervariability sequence called complementarity determining regions (CDR 1, CDR 2 and CDR 3), separated by four framework regions (FR1, FR2, FR2, and FR4) which form a beta-sheet structure and serve as a scaffold to hold the HV regions in position. The C-terminus of each heavy and light chain defines a constant region consisting of one domain for the light chain (CL) and three for the heavy chain (CH1, CH2 and CH3). In some embodiments, the terms “full length” “whole” or “intact” are used in reference to an antibody to mean that it contains two heavy chains and two light chains, optionally associated by disulfide bonds as occurs with naturally-produced antibodies. In some embodiments, an antibody is produced by a cell. In some embodiments, an antibody is produced by chemical synthesis. In some embodiments, an antibody is derived from a mammal. In some embodiments, an antibody is derived from an animal such as, but not limited to, mouse, rat, horse, pig, or goat. In some embodiments, an antibody is produced using a recombinant cell culture system. In some embodiments, an antibody may be a purified antibody (for example, by immune-affinity chromatography).

“Antibody fragments” comprise a portion of an intact antibody, generally the antigen binding or variable region of the intact antibody. Examples of antibody fragments include Fab, Fab′, F(ab′)2, and Fv fragments: diabodies; single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to “polyclonal antibody” preparations which typically include different antibodies directed against different determinants (epitopes), each monoclonal antibody is directed against a single epitope on the antigen. In addition to their specificity, the monoclonal antibodies can frequently be advantageous in that they are synthesized by the hybridoma culture, uncontaminated by other immunoglobulins. The “monoclonal” indicates the character of the antibody as being obtained from a substantially homogeneous population of antibodies, and is not to be construed as requiring production of the antibody by any particular method. The monoclonal antibodies herein specifically include chimeric antibodies and humanized antibodies.

The terms “polypeptide”, “peptide”, and “protein”, as used herein, are interchangeable and are defined to mean a biomolecule composed of amino acids linked by a peptide bond.

The term “subtype” or “serotype” is used herein interchangeably and in reference to a virus, for example, dengue virus and means genetic variants of that virus antigen such that one subtype is recognized by an immune system apart from a different subtype. For example, dengue virus subtype 1 is immunologically distinguishable from dengue virus subtype 2.

As used herein an “antibody pair” refers to two or more antibodies that are specific for two or more different epitopes on the same antigen; for example, two monoclonal antibodies specific for two different epitopes of the same antigen. Exemplary antibody pairs are found in Application No. 62/293,990, entitled Anti-dengue virus NS1 Protein Monoclonal Antibodies filed by Bosch et al. on Feb. 11, 2016.

As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

The word “complex” as used herein refers to the product of a specific binding agent-ligand reaction. Preferably, the term “complex” as used herein refers to a labelled detection antibody bound to its target analyte prior to being detected by, and bound to a capture antibody in a sandwich immunoassay. The combination of detection antibody and capture antibody are also referred to herein as “antibody pairs”.

The term “antigen” refers to a polypeptide or protein that is able to specifically bind to (immunoreact with) an antibody and form an immunoreaction product (immunocomplex). The site on the antigen with which the antibody binds is referred to as an antigenic determinant or epitope.

The term “biological sample,” as used herein, refers to a sample of biological origin, or a sample derived from the sample of biological origin. The biological samples include, but are not limited to, blood, plasma, serum, saliva, cerebral spinal fluid, pleural fluid, milk, lymph, sputum, semen, urine, stool, tear, saliva, needle aspirate, external section of the skin, respiratory, intestinal, or genitourinary tract, tumor, organ, cell culture, cell culture constituent, tissue sample, tissue section, whole cell, cell constituent, cytospin, or cell smear.

As used herein, an “analyte” refers to the material to be detected by use of the assay strips and methods of the present invention. “Analyte” includes but is not limited to: antigens, antibodies, hormones, drugs, proteins associated with a cell (“cell proteins”), secreted proteins, enzymes, cell surface or transmembrane proteins, glycoproteins and other proteins, peptides, and carbohydrates. Preferably the analyte is the NS1 protein of a serotype of dengue virus.

As used herein, a “detection antibody” is, for example, a monoclonal antibody that is conjugated to a detection label and that is specific for a target analyte of interest. As used herein, a “capture antibody” should be understood as an antibody, such as a monoclonal antibody, attached directly or indirectly at the test line of the assay strip of the invention and that is capable of detecting and binding the detection antibody/label complex. The detectable label of the detection antibody includes, but is not limited to: colored particles, such as a metal sol or colloid, preferably gold) wherein such labelled antibody is also referred to herein as the “detection antibody”. The detection and capture antibodies may be bound to, or immobilized on, the assay strip using a variety of techniques known to those in the art, which are amply described in the patent and scientific literature. As used herein, the term “bound” refers to both noncovalent association, such as adsorption, and covalent attachment (which may be a direct linkage between the antibody and functional groups on the support or may be a linkage by way of a cross-linking agent). In certain embodiments, detection antibodies are reversibly associated with the conjugation pad such that they may migrate to the test line as a complex between with their target analyte for detection by the capture antibody present at the test line. In such cases, adsorption can be achieved by contacting the antibody, in a suitable buffer, with the assay strip for a suitable amount of time and allowing the support to dry.

Preferably, the term “about” in the context of any of the above measurements may refer to, for example, +/−5% of a given measurement.

As used herein, the term “specifically binds” refers to the specificity of a binding reagent, e.g., an antibody or an aptamer, such that the binding reagent preferentially binds to a defined target or analyte. A binding reagent “specifically binds” to a target if it binds with greater affinity, avidity, more readily, and/or with greater duration than it binds to other substances. For example, a binding reagent that specifically binds to a target may bind to the target analyte with at least about 10%, at least about 20%, at least about 30%, at least about 40%, at least about 50%, at least about 60%, at least about 70%, at least about 80%, at least about 90% or more, greater affinity as compared to binding to other substances; or with at least about two-fold, at least about five-fold, at least about ten-fold or more of the affinity for binding to a target analyte as compared to its binding to other substances. Recognition by a binding reagent of a target analyte in the presence of other potential interfering substances is also one characteristic of specifically binding. Preferably, a binding reagent, e.g., an antibody or an aptamer, that is specific for or binds specifically to a target analyte, avoids binding to a significant percentage of non-target substances, e.g., non-target substances present in a testing sample. In some embodiments, a binding reagent avoids binding greater than about 90% of non-target substances, although higher percentages are clearly contemplated and preferred. For example, a binding reagent can avoid binding about 91%, about 92%, about 93%, about 94%, about 95%, about 96%, about 97%, about 98%, about 99% and about 99.9% or more of non-target substances. In other embodiments, a binding reagent can avoid binding greater than about 10%, 20%, 30%, 40%, 50%, 60%, or 70%, or greater than about 75%, or greater than about 80%, or greater than about 85% of non-target substances.

As used herein, the term “label” includes a detectable indicator, including but not limited to labels which are soluble or particulate, metallic, organic, or inorganic, and may include spectral labels such as green fluorescent protein, fluorescent dyes (e.g., fluorescein and its derivatives, rhodamine) chemi-luminescent compounds (e.g., luciferin and luminol), spectral colorimetric labels such as colloidal gold, or carbon particles, or colored glass or plastic (e.g., polystyrene, polypropylene, latex, etc.) beads. Where necessary or desirable, particle labels can be colored, e.g., by applying dye to particles. As used herein, the term “colored particle label” includes, but is not limited to colored latex (polystyrene) particles, metallic (e.g., gold) sols, non-metallic elemental (e.g., Selenium, carbon) sols and dye sols.

Preferably the invention provides a lateral flow multiplexed assay strip comprising:

(a) a porous matrix that enables capillary flow along the matrix;
(b) a sample pad at the upstream end of the matrix that provides absorption of a liquid sample;
(c) a conjugation pad downstream from the sample pad, wherein said conjugation pad comprises two or more different detection antibodies, wherein each detection antibody is specific for a different target analyte and wherein each detection antibody is conjugated to at least one detectable label that is different from the detectable label of any other detection antibody, wherein each unique label comprises a different spectral emission and wherein each detection antibody is capable of forming a complex with its target analyte;
(d) a single test area downstream from the conjugation pad wherein the test area comprises at least two different capture antibodies immobilized on the single test area, wherein each capture antibody is specific for a different target analyte;
(e) an optional control area downstream from the single test area, wherein the control area comprises a positive or negative control reagent; and
(f) an optional wick pad, downstream of the positive control area wherein said wick pad provides absorption of excess reagents and maintains a lateral flow along the support.

Preferably the invention provides lateral flow multiplexed assay strip comprising:

(a) a porous matrix that allows capillary flow along the matrix;
(b) a sample pad at the upstream end of the matrix that provides absorption of a liquid sample upon contact with the liquid sample wherein the liquid sample comprises two or more different detection antibodies, wherein each detection antibody is specific for a different target analyte that may be present in the sample and wherein each detection antibody comprises at least one detectable label that is different from the detectable label of any other detection antibody, wherein each label comprises a unique spectral emission and wherein each labelled detection antibody is capable of forming a complex with its target analyte;
(c) a single test area downstream from the conjugation pad wherein the test area comprises at least two different capture antibodies immobilized on the single test area, wherein each capture antibody is specific for a different target analyte;
(d) an optional control area downstream from the single test area, wherein the control area comprises a positive control;
(e) an optional wick pad, downstream of the positive control area wherein said wick pad provides absorption of excess reagents and maintains a lateral flow along the matrix; and
(f) an optional backing or housing for the porous matrix.
In this format, the labelled detection antibody is allowed to bind to its target analyte in the biological sample instead of being bound to the assay strip at the conjugation pad.

The present invention is useful for highly sensitive detection of multiple analytes in a sample, for example, wherein the analytes are present in the sample at concentrations ranging from about 1 ng/ml to about 20 ug/ml, preferably, about 1 ng/ml to about 15 ug/ml, preferably about 1 ng/ml to about 1 ug/ml. Exemplary analytes include markers for diseases or conditions such as infectious diseases and parasitic diseases including, but not limited to dengue virus.

The biological sample is preferably a bodily fluid that may contain a target analyte of interest. This fluid may be serum whole blood, plasma, colostrum, milk, saliva, tears, or urine sample from a human or other animal species.

Preferably, the multiplexed assay strip is in form of a strip may be made of, for example, a porous matrix, such as a flat piece of nitrocellulose or other support structure that may be coated with or impregnated or otherwise including nitrocellulose or any other polymer suitable for a chromatographic process. The strip may be in a form of a plain narrow piece, or it may, for example, be coil-shaped in order to increase its length in the same volume, and hence, improve separation of the components of the liquid sample. Preferably the strip is small and portable such that it requires only a small amount of sample for testing but is large enough to provide a separate test area for detection, for example the dimensions may be about 2 cm long and 3 mm wide. The assay strip may preferably include a backing to provide rigidity to the assay strip (e.g., plastic) and/or a housing (e.g., plastic) that can optionally cover portions of the porous matrix to protect the matrix and any antibodies bound thereto during storage and transport prior to use and during use. Such housing may be removable or remain in place so long as the sample pad portion of the matrix is accessible to sample and results at the single test area can be easily read. Lateral flow assay devices that are capable of being handheld are known in the art. For ease of reference, the terms “assay support” and “assay strip” may be used interchangeably herein however it is understood that the assay strip need not necessarily be a strip but may take other shapes.

The assay of the invention preferably makes use of a conjugate comprising a protein such as antibody bound to a label component. Any detectable label recognized in the art as being useful in various assays could be used in the present invention. In particular, the detectable label component can include compositions detectable by spectroscopic, photochemical, biochemical, immunochemical, or chemical means. The label component thus produces a detectable signal. Exemplary labels include fluorescent dyes, chemiluminescent compounds, radioisotopes, electron-dense reagents, enzymes, or colored particles (such as a metal sol or colloid, preferably gold). The label component can generate a measurable signal, such as radioactivity, fluorescent light, color, or enzyme activity, which can be used to identify and quantify the amount of label bound to a test site. Thus, the label component can also represent the presence of a particular antigen bound thereto.

A suitable label depends on the intended detection methods. The label can be a direct label or an indirect label. A direct label can be detected by an instrument, device or naked eyes without further step to generate a detectable signal. A visual direct label, e.g., a gold or latex particle label, can be detected by naked eyes. An indirect label, e.g., an enzyme label, requires further step to generate a detectable signal. In some embodiments, the label is a soluble label, such as a colorimetric, radioactive, enzymatic, luminescent or fluorescent label. Depending on the specific configurations, the labels such as colorimetric, radioactive, enzymatic, luminescent or fluorescent label can be either a soluble label or a particle or particulate label.

Preferably, the detectable label having a unique spectral emission includes, but is not limited to, noble metal nanoparticles (NP) such as gold or silver nanoparticles, colored latex beads, magnetic particles, carbon nanoparticles, selenium nanoparticles, quantum dots, up converting phosphors, organic fluorophores and enzymes. Preferably the detectable labels provide a direct spectral signal at the completion of the assay such as the color detectable color from metal nanoparticles. Color release from an enzyme conversion for example requires an extra step to produce a spectral signature which is preferably avoided.

Optionally, the assay strip may comprise a control line. The control line is used as a control to ensure the assay reagents are working and that lateral flow is occurring. The controls for detecting the formation and sufficient migration of the particle-labeled, analyte-specific reagent can take different forms. The control may be a positive or negative control as is well understood in the art. Preferably, the control is a positive control and comprises an antibody capable of binding the Fc region that detects the Fc region of antibodies coupled to the NP.

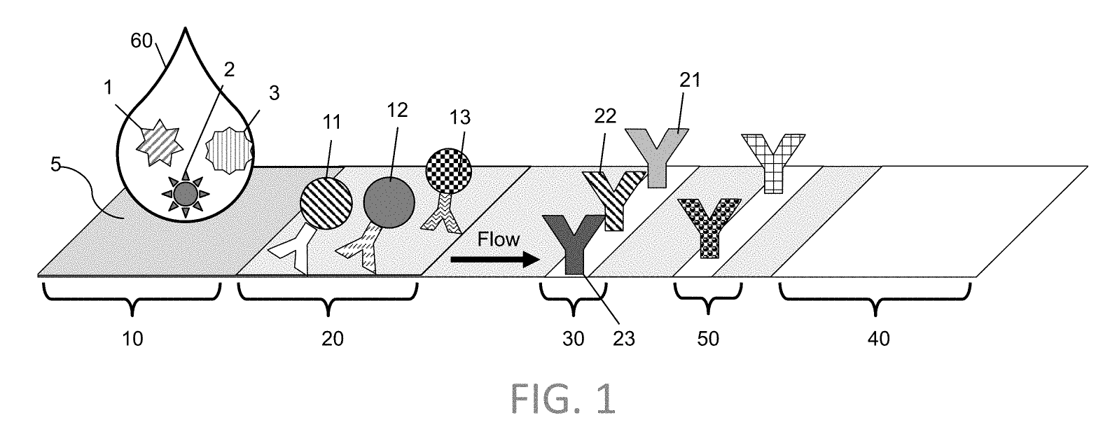

FIG. 1 shows the general architecture of a preferred lateral flow assay strip of the present invention. The assay strip 5 has a sample pad 10 capable of absorbing a liquid sample 60 potentially containing one or more target analytes 1, 2, 3. The assay strip also comprises a conjugate pad 20 comprising labeled antibodies 11, 12, 13, each specific for different analytes 1, 2 and 3. The assay strip also comprises a single test area 30 comprising at least two different antibodies, 21, 22, 23, each specific for a different analyte 1,2 3. The assay strip may also include a control line 50 comprising, for example an antibody capable of detecting the Fc region of the conjugate, and wicking pad 40.

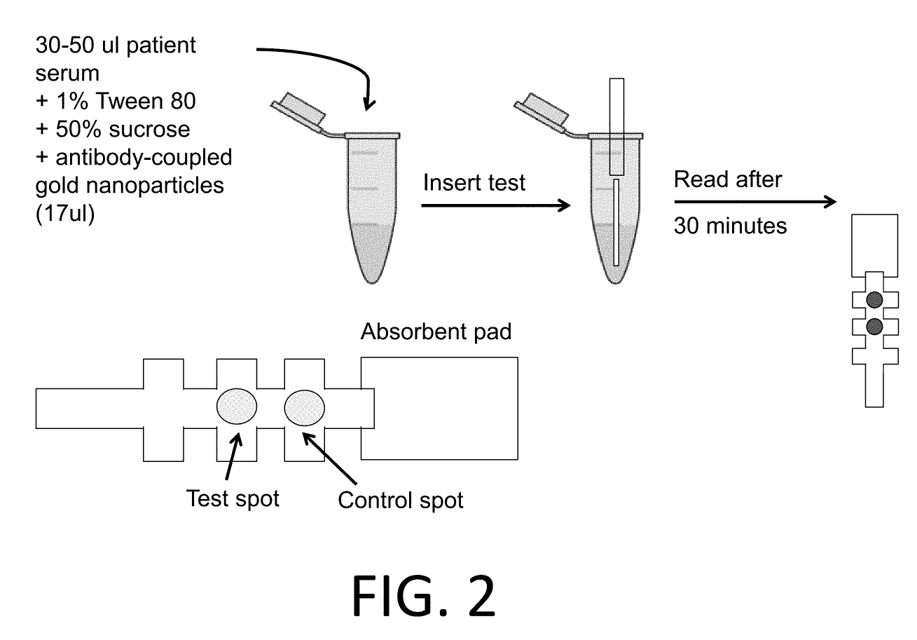

Alternatively, other assay formats will be apparent to those of skill in the art. For example, the assay strip of FIG. 2 wherein instead of a conjugate pad, the assay strip is contacted with a liquid sample comprising labelled detection antibody and potential analyte as is shown in FIG. 2. In this format, the labelled detection antibody forms a complex with its target analyte in the liquid sample which is thereafter contacted with the assay strip of the invention. This configuration shown in FIG. 2 is also referred to herein as a “dipstick” format. The complex wicks through the pad to test single line comprising at least two different antibodies, each specific for a different analyte shown and any flow through is contacted with the control line as shown in FIG. 2.

The invention also provides kits comprising the lateral assay strip of the invention.

Preferably a kit comprises an assay strip of the invention, a vial or container such as a test tube or Eppendorf tube to hold a liquid sample which may also comprise labelled detection antibody depending on the assay format chosen. Preferably, the kit may also contain appropriate buffers or other liquids to be combined with a sample suspected of containing an analyte and which may assist in stabilizing the analyte or forming an aqueous solution or liquid suspension containing the analyte. Preferably, the kit may also comprise additional reagents or buffers or medical equipment such as sterile syringes, for obtaining or collecting the sample, a vial or other container for holding and/or storing the sample and/or one or more reagents and buffers and optionally, a standard that is compatible with the detection label being used to determine the presence or absence of a detectable label on the single test area of the assay strip.

The invention also provides an assay system comprising the assay strips or kits of the invention, and a reader apparatus for detecting signal from the assay. Preferably the reader apparatus such as a colorimetric sensor is designed to measure emitted light from analyte trapped by capture antibody at the single test area of the assay strip. Optionally, the apparatus is designed to spectrally filter an emission signal to selectively detect light emitted by the corresponding spectrally encoded detection antibody. Optionally, the reader apparatus is adapted to read an assay dynamically in real time as the assay develops or statically at a selected time point after initiation of the assay.

Recent advances in consumer electronics and wireless communication devices have cultivated a transformation in biomedical imaging, sensing and diagnostics. By leveraging the power of semiconductor sensor chips and carry-on optics, mobile-phone based devices have become a versatile microscopy/nanoscopy and sensing platform for a wide range of applications, including blood analysis, bacteria detection, single-virus imaging, DNA imaging and sizing, chemical sensing, and biomarker detection, among others.

Therefore, the invention provides for the analysis of an assay strip of the invention by way of capturing an image of the assay on the camera built into the mobile phone, an optional tool being provided to enable the assay to be positioned an appropriate distance from the phone camera. A software application on the phone can then analyze the captured image to determinate a qualitative or quantitative outcome of the assay. In many examples, the test will require no modification of the phone hardware and is thus a convenient and cheap technique for analyzing an assay. In other embodiments, other items such filter(s) and/or additional light source(s) may be provided.

The present invention also provides a multiplex assay method for detecting multiple target analytes on a single test area comprising the steps of:

applying a sample to the sample pad of the lateral flow multiplexed assay system of the invention; and

detecting the unique spectral emissions present at the single test area using a colorimetric reader.

Preferably, the colorimetric reader is a mobile phone having a suitable software application capable of providing, for example, a Red Green Blue (RGB) analysis of the image of the completed assay. Such software is known in the art and includes but is not limited to:

ImageJ analysis software suitable for use with the present invention is known in the art and includes, but is not limited to ImageJ software available for downloading through the NIH website.

Other options for colorimetric readers include lap top computers linked to a scanner. Also, a photo of the assay strip may be acquired with the mobile phone and sent to a server via mobile internet that is in turn linked to a spectrometer or other device capable of reading the color intensity of the photo.

The invention is further illustrated by the following non-limiting examples.

### EXAMPLES

### Example 1: Multiplexed Lateral Flow Point of Care (POC) Diagnostic

Here, we exploit the size-dependent optical properties of silver nanoparticles (Ag NPs) to construct a multiplexed lateral flow POC sensor. We conjugate triangular plate-shaped AgNPs of varying sizes to antibodies that bind to specific biomarkers, and thus use NP color to distinguish among three pathogens that cause a febrile illness. Because positive test lines can be imaged by eye or by using a mobile phone camera, the approach is adaptable to low-resource, widely deployable settings.

Noble metal NPs are attractive for lateral flow POC diagnostics because they are visible without an external excitation source or emission sensor, and unlike small-molecule dyes, resist photo bleaching. In addition, NP molar extinction coefficients typically exceed those of dyes by several orders of magnitude (10⁸ vs. 10⁴ M⁻¹cm⁻¹). Huang et al., Nanomedicine (2007) 2:681693. NP surface area is large and available for biofunctionalization with an antibody or nucleic acid aptamer that can bind to specific targets. More importantly, the colorimetric properties of NPs can be tuned by varying shape and/or size. Triangular plate-shaped silver NPs (AgNPs) have narrow absorbances that are tunable through the visible spectrum, Sherry et al., Nano Lett. (2006) 6:2060-2065 resulting in easily distinguishable colors. AgNPs were synthesized using a seed-mediated growth method. Homan et al, ACS Nano (2012) 6:641-650. Growth of the yellow seeds to large AgNPs resulted in color changes from yellow to orange, red, blue, and green, with expected absorption spectrum shifts. Growth also resulted in a morphology change from spherical particles to triangular nanoplates. The AgNP colors are evident and distinguishable from one another when applied to paper and dried (FIG. 1g). TEM imaging and dynamic light scattering confirmed that the NPs had distinct sizes, with mean diameters of D_{orange}=30±7 nm, D_{red}=41±6 nm, and D_{green}=47±8 nm.

AgNPs were prepared for lateral flow chromatography by conjugating antibodies to the NPs. Combining antibodies with AgNP in solution results in antibody binding to the AgNP mainly by electrostatic adsorption. Antibodies recognizing dengue virus (DENV) NS1 protein, Yellow Fever Virus (YFV) NS1 protein, and Ebola virus, Zaire strain (ZEBOV) glycoprotein GP were used. Ebola belongs to the Filoviridae virus family, while DENV and YFV are members of the Flaviviridae family. Our goal was to demonstrate detection without cross-contamination on the sample pad. We choose pairs of monoclonal antibodies directed against DENV NS1 denatured protein (antibodies F4.24 and 8H7.G10), YFV NS1 as well as ZEBOV glycoprotein (GP). Orange AgNPs, red AgNPs, and green AgNPs were conjugated with anti-YFV NS1 monoclonal antibody (mAb), anti-ZEBOV GP mAb, or anti-DENV NS1 mAb, respectively. After conjugation, AgNP surfaces were backfilled with thiolated PEG (mPEG-SH, MW=5,000) to enhance conjugate stability. Upon conjugation, the mean hydrodynamic size increased by ˜50 nm, and the negative charge decreased, suggesting successful functionalization of the AgNPs with the antibodies.

As shown in the schematic (FIG. 1), the components of the lateral flow chromatography assay used in this experiment included a sample pad (SP), conjugate pad (CP), nitrocellulose membrane (NC), and wick/absorbent pad. Each nitrocellulose fluidic pathway has four detection areas: a blank area to assess background binding to the NC, a second blank area that can be used to assess non-specific binding to an unrelated antibody, test area, and positive control area (bottom to top). The final component is the absorbent pad, which wicks fluid by capillary action. Conjugated AgNP-Ab were pipetted onto conjugate pads (CP) of the lateral flow assemblies, yielding orange, red, and green colors. The fourth CP is brown because it was loaded with a mixture of all three colored AgNPs. A “sandwich” is formed when the viral protein ligand (NS1 or GP) is bound by both the antibody conjugated to the NP, and to the capture antibody loaded onto the test area of the nitrocellulose membrane. In a typical run, the sample solution containing the antigen (NS1 protein of DENV or YFV; GP of ZEBOV) is loaded into the sample pad. The liquid migrates through the CP, where the antigen binds to the AgNP-Ab. The AgNP-Ab/antigen complex then wicks through the strip by capillary action. As specific AgNP-Ab/antigen complexes flow through the strip, they are captured by the antibodies printed at the test line, creating a colored band at the test detection area. A positive control detection area is essential to demonstrate that the test ran completely and that the reagents were functional. We used as a positive control an antibody that detects the Fc region of antibodies coupled to the NP. A colored test area is present at the positive control area due to the antibody on the AgNPs binding to an anti-immunoglobulin antibody loaded at the positive control detection area. Excess unbound AgNP-Ab flow into the absorbent pad.

A limit of detection analysis (LOD) for each of the three viral proteins was assessed. Antibody pairs (conjugated to the AgNP or loaded onto the test detection area) were specific to the YFV NS1 protein (orange), Ebola GP (red) or Dengue NS1 protein (green). The positive control detection area was loaded with anti-mouse immunoglobulin antibody to capture any mouse anti-human antibodies. Equal volumes of solutions containing the indicated protein concentrations were spiked into human serum (human male AB plasma purchased from Sigma Aldrich) loaded onto the sample pads (0-500 ng/ml). Positive signals at the positive control detection area confirmed that the test ran to completion and that AgNP-Ab were functional. In the absence of antigen, the test line was blank, indicating that AgNP-Ab-antigen binding is specific, and that non-specific adsorption of the AgNP-Ab to the test line was undetectable. LODs for YFV, DENV, and ZEBOV proteins were all in the range of 150 ng/mL. The maximal dengue NS1 serum concentration has been estimated to be 15-50 ug/ml (Young et al, J. Clin. Microbiol. (2000), 38:1053-1057 and Lcon et al, J. Clin. Microbiol, (2002) 40:376-381). Therefore, our observed LOD is estimated to be 100-300× lower than this value, providing a significant window for early detection at lower serum NS1 concentrations. For DENV NS1, the LOD is more sensitive than the reported LOD of NS1 protein detected by the paper based lateral flow device (Wang et al, Adv. Healthcare Mater, (2014) 3:187-196). The values for ZEBOV GP and YFV NS1 levels in human serum after infection have not been reported. Three independent measurements made from separate conjugation and antigen were repeated for LOD and the results were evaluated by ImageJ (an open source java based software image analysis program that is available for download from the NIH website). RGB analysis using ImageJ further distinguished and characterized the AgNP (orange NPs: R=162±15, G=79±10, B=60±4; red NPs: R=219±5, G=101±8, B=151±7; green NPs: R=104±13, G=111±15, B=96±14).

Multiplexed detection was explored using two different platforms. First, conjugate pads were prepared by loading with a mixture of orange, red, and green AgNPs conjugated with antibodies directed against YFV NS1 (orange), ZEBOV GP (red), and DENV NS1 (green), respectively. The second (“capture”) antibody directed against the viral protein was loaded separately onto individual detection areas of the fluidic paths. The sample pad of strip 1 was loaded with human serum only. The signal at each of the detection areas is undetectable, demonstrating very low background binding signal. When YFV NS1 (strip 2), ZEBOV GP (strip 3) or DENV NS1 (strip 4) protein was applied to the sample pad, the orange, red, and green signals were observed only at the corresponding test detection area where the respective capture antibodies were loaded (strips 2-4). Again, background/non-specific binding at other test detection areas was minimal. When all three proteins were combined and applied to the sample pad (strip 5), orange, red, and green signals were observed at the position corresponding to the respective capture antibodies. The control detection area of each test was brown due to the presence of orange, red, and green AgNPs. RGB values of the control detection area indicated that all three colors of AgNPs were present. The clear signal and low background/non-specific binding in each of the tests demonstrate effective multiplexed detection using mixtures of conjugated AgNPs with single capture antibodies loaded at the detection areas.

We next tested multiplexing by loading a single membrane detection area with a mixture of capture antibodies. The rationale of this approach is to reduce the number of test detection areas from three to one. The three monoclonal capture antibodies were mixed at equimolar concentrations and then printed onto a single detection area on the test strip. Conjugate pads were again loaded with a mixture of the three AgNP-Ab conjugates. The predicted result of the experiment was that the single test detection area would be orange if YFV NS1 were present, red if ZEBOV GP were present, or green if DENV NS1 were present. Indeed, data are consistent with predicted results. When all three proteins were mixed, the detection area was brown due to the mixture of orange, red, and green nanoparticles. RGB analysis was used to quantify the test line colors, and the results showed that the values were similar to those of the individual AgNPs used in the lateral flow. The RGB value of each test is plotted in three axes (R, G, and B). Each antigen detection forms an ellipse and none of ellipses overlap with the others, indicating that non-specific binding was not detected and that AgNP-Abs could bind without crossover reactivity. These data strongly suggest that multiplexed detection can be achieved in a single test area, which can facilitate miniaturization by increasing the number of targeted antigens in a given strip. This detection configuration could reduce test strip dimensions and simplify device design, potentially reducing material costs.

In summary, AgNP optical properties can be utilized for multiplexed POC diagnostics for infectious disease using their size-tunable absorption spectra. Distinguishing the color of the test lines can distinguish between different biomarkers, which can be achieved in a variety of formats including mobile phone apps Shen et al., Lab Chip (2012) 12:4240-4243; Vashist et al., Anal. Bioanal. Chem. (2014) 406:3263-3277; and Zhen et al, Nat. Biotechnol (2005) 23:1294-1301). LODs for the biomarkers of each disease were 150 ng/mL. This type of design is ready for multiplexed detection with reduced device dimensions and cost.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention

What is claimed is:

1. A lateral flow multiplexed assay strip comprising:
(a) a porous matrix that enables capillary flow along the matrix;
(b) a sample pad at the upstream end of the matrix that provides absorption of a liquid sample;
(c) a conjugation pad downstream from the sample pad, wherein said conjugation pad comprises two or more different detection antibodies, wherein each detection antibody is specific for a different target analyte and wherein each detection antibody is conjugated to at least one detectable label that is different from the detectable label of any other detection antibody, wherein each unique label comprises a different spectral emission and wherein each detection antibody is capable of forming a complex with its target analyte;
(d) a single test area downstream from the conjugation pad wherein the single test area comprises at least two different capture antibodies immobilized on the single test area, wherein each capture antibody is specific for a different target analyte;
(e) an optional control area downstream from the single test area, wherein the control area comprises a positive or negative control reagent;
(f) an optional wick pad, downstream of the positive control area wherein said wick pad provides absorption of excess reagents and maintains a lateral flow along the porous matrix; and
(g) an optional backing or housing for the porous matrix.

2. The multiplexed assay strip of claim 1, wherein the porous matrix comprises nitrocellulose.

3. The multiplexed assay strip of claim 1, wherein the detectable label is selected from: gold nanoparticles, colored latex beads, carbon nanoparticles, selenium nanoparticles, silver nanoparticles quantum dots, up converting phosphors, organic fluorophores.

4. The multiplexed assay strip of claim 1, wherein the positive control comprises an antibody specific for the Fc portion of an antibody.

5. The multiplexed assay strip of claim 1, wherein the target analyte is present in a biological sample.

6. The multiplexed assay strip of claim 1, wherein the matrix comprises nitrocellulose.

7. A lateral flow multiplexed assay system comprising the multiplexed assay strip of claim 1 and a colorimetric sensor.

8. The assay system of claim 7, wherein colorimetric sensor detects red-green-blue (RGB) values.

9. The assay system of claim 7, wherein the colorimetric sensor is a mobile phone comprising an RGB color analysis application installed thereon.

10. A multiplex assay method for detecting multiple target analytes on a single test area comprising the steps of:
applying a sample to the sample pad of the lateral flow multiplexed assay system of claim 7; and
detecting the RGB values present at the single test area using the colorimetric sensor.

11. The method of claim 10, wherein the sample is a biological sample.

12. The method of claim 10, wherein at least one of the target analytes is derived from a virus.

13. A lateral flow multiplexed assay strip comprising:
(a) a porous matrix that allows capillary flow along the matrix;
(b) a sample pad at the upstream end of the matrix that provides absorption of a liquid sample upon contact with the liquid sample wherein the liquid sample comprises two or more different detection antibodies, wherein each detection antibody is specific for a different target analyte that may be present in the sample and wherein each detection antibody comprises at least one detectable label that is different from the detectable label of any other detection antibody, wherein each label comprises a unique spectral emission and wherein each labelled detection antibody is capable of forming a complex with its target analyte;
(c) a single test area downstream from the conjugation pad wherein the single test area comprises at least two different capture antibodies immobilized on the single test area, wherein each capture antibody is specific for a different target analyte;
(d) an optional control area downstream from the single test area, wherein the control area comprises a positive control;
(e) an optional wick pad, downstream of the positive control area wherein said wick pad provides absorption of excess reagents and maintains a lateral flow along the matrix; and
(f) an optional backing or housing for the porous matrix.

14. The multiplexed assay strip of claim 13, wherein the matrix comprises nitrocellulose.

15. The multiplexed assay strip of claim 13, wherein the detectable label is selected from: gold nanoparticles, colored latex beads, carbon nanoparticles, selenium nanoparticles, silver nanoparticles quantum dots, up converting phosphors, organic fluorophores.

16. The multiplexed assay strip of claim 13, wherein the positive control comprises an antibody specific for the Fc portion of an antibody.

17. The multiplexed assay strip of claim 13, wherein the target analyte is present in a biological sample.

18. The multiplexed assay strip of claim 13, wherein the matrix comprises nitrocellulose.

19. A multiplexed assay system comprising the assay strip of claim 13 and a colorimetric sensor.

20. The multiplexed assay system of claim 19, wherein colorimetric sensor detects red-green-blue (RGB) values.

21. The multiplexed assay system of claim 19, wherein the colorimetric sensor is a mobile phone comprising an RGB color analysis application installed thereon.

22. A multiplex assay method for detecting multiple target analytes on a single test area comprising the steps of:
contacting a liquid sample with the sample pad of the lateral flow multiplexed assay system of claim 19; and
detecting the RGB values present at the single test area using the colorimetric sensor.

23. The method of claim 22, wherein the liquid sample is a biological sample.

24. The method of claim 22, wherein at least one of the target analytes is derived from a virus.

25. A kit comprising the multiplexed assay strip of claim 1 and a container for holding a sample.

26. A kit comprising the multiplexed assay strip of claim 13 and a container for holding a sample.

27. The multiplexed assay strip of claim 1, wherein the porous matrix is in the form of a strip.

28. The multiplexed assay strip of claim 13, wherein the porous matrix is in the form of a strip.

29. The multiplex assay strip of claim 13, wherein the assay strip is in a dipstick format and the sample pad is contacted with sample by dipping the sample pad of the assay strip into a container holding the sample.
